(19) United States
(12) Patent Application Publication
(10) Pub. No.: US 2013/0108560 A1
(43) Pub. Date: May 2, 2013

(54) Chemical Treatments for the Disruption of Dental Plaque Biofilms and Related Methods

(71) Applicants: Robert Davidson, Woodland Hills, CA (US); Eric Allen, Camarillo, CA (US); Ed Maliski, Thousand Oaks, CA (US)

(72) Inventors: Robert Davidson, Woodland Hills, CA (US); Eric Allen, Camarillo, CA (US); Ed Maliski, Thousand Oaks, CA (US)

(21) Appl. No.: 13/666,746

(22) Filed: Nov. 1, 2012

Related U.S. Application Data
(60) Provisional application No. 61/554,161, filed on Nov. 1, 2011.

(51) Int. Cl. A61K 8/44; A61Q 11/00; C07C 321/04; C07C 229/36; C07C 229/08; C07D 209/20

(52) U.S. Cl. 424/54; 562/575; 548/496; 562/559; 562/445

(57) ABSTRACT

A thin film composition for oral administration that adheres to and dissolves in a mouth of a user, wherein the thin film is a single layered water-soluble solid comprising at least one D-amino acid contained in a plurality of hydrophobic carriers dispersed throughout the thin film. The hydrophobic carriers comprise oil and the composition further comprises a phospholipid, an emulsifier, and a water soluble polymer. The preferred D-amino acids are D-leucine, D-tryptophan, D-methionine, and D-tyrosine. A method of reducing dental plaque in a subject entails placing the thin film composition contemplated herein into a mouth of the subject.

### CROSS-REFERENCE TO RELATED APPLICATION

This application seeks priority to U.S. Provisional Patent Application Ser. No. 61/554,161 filed on Nov. 1, 2011 entitled, “Chemical Treatments for the Disruption of Dental Plaque Biofilms” the contents of which are incorporated by reference herein.

### FIELD OF INVENTION

The present invention generally relates to the technical field of dental plaque removal. More particularly, the present invention is in the technical field of chemical treatments for the disruption, destruction, and removal of dental plaque.

### BACKGROUND OF THE INVENTION

Dental plaque is one form of a biofilm produced by colony-forming bacteria that inhabit the human oral microbiome. Dental plaque/biofilm (hereinafter, simply “plaque”) enables bacterial colonies to adhere to tooth surfaces, and shields the bacterial colonies from disruption/destruction factors in the oral environment. Acid produced by these colony-forming bacteria creates dental caries (commonly referred to as “tooth decay”). Once formed, dental plaque calcifies due to the accumulation of salival minerals, resulting in rock-hard calculus or tartar, which is difficult to remove except by mechanical means including sharp metallic periodontal scalers and ultrasonic disruption. Tartar is linked to inflammation of the periodontium, which leads to a more serious condition called gingivitis. Gingivitis has been linked to cardiovascular disease. Inflammation also leads to periodontitis which is a progressive inflammation of the periodontium involving progressive loss of dental bone which ultimately leads to tooth loss.

Dental plaque is typically removed mechanically, often by dental professionals, as part of regularly scheduled visits. Some chemical treatments exist but are limited in effectiveness, and are usually strong oxidizing agents such as hydrogen peroxide, organic peroxyacids, sodium chlorite (chlorine dioxide precursor), and sodium hypochlorite (chlorine bleach).

There is need for effective, safe, and convenient treatments for dental plaque that can be used outside of professional dental clinics. This treatment would eliminate or reduce the need for mechanical removal by dental professionals, both increasing overall dental health and reducing the overall costs of dental care.

Animals, particularly companion animals such as dogs and cats, are subject to the same dental plaque infection and inflammation as humans, and can therefore benefit from safe and effective treatments for dental plaque administered outside a clinical or veterinary medical setting.

### SUMMARY

The present invention is directed to an apparatus that satisfies the need for a more desirable treatment and method to promote the disruption of plaque such that the plaque will be removed from teeth through ordinary motions of the mouth and tongue. The present invention also reduces or eliminates the need for mechanical or ultrasonic removal of plaque. Such plaque disruption reduces or eliminates dental caries, tooth decay, periodontal diseases such as periodontitis and gingivitis, halitosis, and tooth discoloration.

The present invention is directed to a class of oral thin films (“OTF”) 8 that contain agents that cause the disruption of dental plaque. These OTFs 8 comprise any or all of the following active ingredients: D-amino acids, D,L-amino acids, flavorings, sweeteners, bitter-blockers, pectin, grapeseed and other natural and modified oils, fatty acids, fatty esters, phospholipids, talc, binders, other micelle and liposome-forming materials, other encapsulating materials, colorants, sugars, oligosaccharides, starches, anti-oxidants, and water.

In particular, the present invention is directed to an OTF 8 composition for oral administration that adheres to and dissolves in a mouth of a user. The OTF 8 is a water-soluble monolayer solid comprising at least one D-amino acid. In one embodiment, the D-amino acid is approximately 0.1% to approximately 4% w/w of the composition. In a preferred embodiment, the composition includes D-leucine, D-tryptophan, D-methionine, and D-tyrosine. In a related embodiment, the composition has about 0.01% to about 1.0% w/w D-leucine; about 0.01% to about 1.0% w/w D-tryptophan; about 0.01% to about 1.0% w/w D-methionine; and about 0.01% to about 1.0% w/w D-tyrosine.

In another embodiment, the D-amino acid is contained in a plurality of hydrophobic carriers dispersed throughout the OTF 8. The hydrophobic carriers are preferably made of oil, and preferably grapeseed oil. In addition, the composition further contains a phospholipid, an emulsifier, and a water soluble polymer.

The OTF 8 composition contains grapeseed oil as approximately 0.5% to approximately 4% w/w of the composition. The phospholipid is approximately 0% to approximately 4% w/w of the composition, the emulsifier is approximately 0% to approximately 6% w/w of the composition, and the water soluble polymer is approximately 4% to approximately 9% w/w of the composition.

In preferred embodiment, the phospholipid is hydroxylated lecithin, the emulsifier is glycerin, and the water soluble polymer is pectin. Flavorings are also added to the composition, with sweetener being approximately 1% to about 7% w/w of the composition and a flavoring being approximately 1% to about 5% w/w of the composition. The sweetener is preferably one or more of acesulfame potassium, sucrose, and sucralose.

These and other objects, aspects, and advantages of the present invention will be better appreciated in view of the following Detailed Description of Embodiments.

### BRIEF DESCRIPTION OF THE DRAWINGS

FIG. 1 is a diagram illustrating an oral thin film;

FIG. 2 is a diagram illustrating a phospholipid molecule;

FIG. 3 is a diagram illustrating active ingredients within a micelle; and

FIG. 4 is a diagram of a liposome suspended in an aqueous solution.

### DETAILED DESCRIPTION OF EMBODIMENTS

In the Summary above and in the Detailed Description of Embodiments, reference is made to particular features (including method steps) of the invention. It is to be understood that the disclosure of the invention in this specification includes all possible combinations of such particular features, regardless of whether a combination is explicitly described. For example, where a particular feature is disclosed in the context of a particular aspect or embodiment of the invention, that feature can also be used, to the extent possible, in combination with and/or in the context of other particular aspects and embodiments of the invention, and in the invention generally.

The term “comprises” is used herein to mean that other features or steps are optionally present. When reference is made herein to a method comprising two or more defined steps, the steps can be carried in any order or simultaneously (except where the context excludes that possibility), and the method can include one or more steps which are carried out before any of the defined steps, between two of the defined steps, or after all of the defined steps (except where the context excludes that possibility).

This invention may be embodied in many different forms and should not be construed as limited to the embodiments set forth herein. Rather, these embodiments are provided so that this disclosure will be thorough and complete, and will convey the scope of the invention to those skilled in the art.

Embodiments of the invention are described herein in connection with oral thin films, or physiologically acceptable films particularly adapted to adhere to and dissolve in a person's mouth to deliver at least one active ingredient that causes the disruption, reduction, or elimination of dental plaque. It is to be understood, however, that the invention is not limited to the specific size, shape, or formulations described herein. The invention may be adapted as desired for use with any oral thin film comprising D-amino acids and/or D,L-amino acids administered for the disruption, reduction, or elimination of dental plaque.

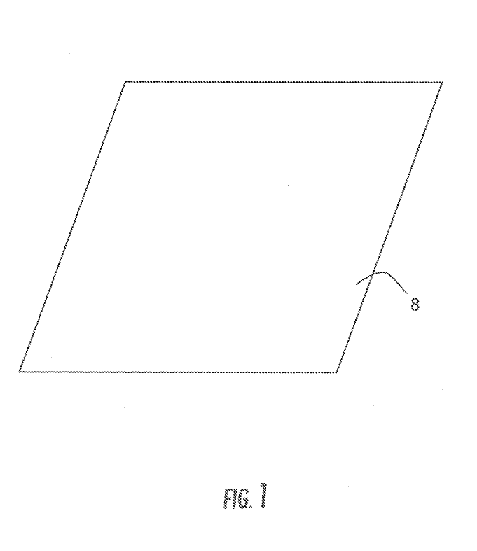

As illustrated in FIG. 1, the present invention relates to a class of oral thin films 8 that are incorporated into products that when administered orally disrupt dental plaque. Such oral thin film 8 products may be used as a pre-treatment for fluoride treatment or in the absence of fluoride treatment. Fluoride may alternatively be incorporated into the oral thin film. Additionally, products utilizing such oral thin films 8 that are designed for humans could easily be adapted for use in animals. These oral thin films 8 contain at least one of the following, without limitation: D-amino acids, D,L-amino acids, flavorings, sweeteners, acesulfame potassium, aspartame, sucralose, bitter-blockers, pectin, grapeseed and other oils, glycerin, talc, binders, other micelle and liposome-forming materials, other encapsulating materials, colorants, tooth-whitening agents, fluoride, starches, anti-oxidants, and water.

The active ingredients in the oral thin film 8 may also be incorporated into chewing gum, oral gels, toothpastes, mouthwashes, oral sprays, lozenges, candies, lollipops, orally disintegrating tablets, dissolving film, gelatin-based chewy candy (“gummi” candy), and similar types of more slowly dissolving medicament-delivering vehicles that contain materials which reduce or eliminate dental plaque.

As used herein, the term “pharmaceutically active ingredient” or “active ingredient” means an ingredient in the composition that produces a physiological effect in the user. Preferred active ingredients include D-amino acids and mixtures containing D/L amino acids. The active ingredient is at least one D-amino acid, such as D-alanine, D-arginine, D-asparagine, D-aspartic, D-cysteine, D-glutamine, D-glutamic, D-histidine, D-isoleucine, D-leucine, D-lysine, D-methionine, D-phenylalanine, D-proline, D-serine, D-threonine, D-tryptophan, D-tyrosine, and D-valine. In a preferred embodiment, the invention comprises a mixture of D-leucine, D-tryptophan, D-methionine, and D-tyrosine.

The contemplated dosage of active ingredient varies as long as they are in amounts sufficient to provide anti-plaque properties. The preferred dose is about up to 250 mg of D-amino acids. In a preferred embodiment, a plurality of D-amino acids are used as active ingredients, the dose of each D-amino acid being about 10 mg to about 75 mg. In a more preferred embodiment, the active ingredients comprise 0.6-12.0 mg D-leucine, 0.9-18.0 mg D-tryptophan, 0.7-14.0 mg D-methionine, and 0.8-16.0 mg D-tyrosine per serving. The preferred amount of active ingredient for manufacturing the compositions contemplated herein is about 0.01% to about 5.0% w/w and preferably in amounts of about 0.02% to about 1.0% w/w, even more preferably about 0.03% to about 0.05% w/w.

In one embodiment of the present invention active ingredients are at least partially contained in hydrophobic carriers that are dispersed in a water soluble polymer or mucoadhesive polymer. Preferably, the hydrophobic carriers are either micelles, liposomes, or oil droplets in a colloidal suspension. The liposomes and/or micelles are made of one or more phospholipids. Referring to FIG. 2, a phospholipid 10 is a type of amphiphilic lipid. A typical phospholipid 10 has a hydrophilic phosphate head group 12 and a hydrophobic tail 14.

Referring to FIG. 3 a micelle 20 is formed from amphiphilic molecules, such as phospholipids 10. When dispersed in an aqueous solution, the hydrophilic head groups 12 form a hydrophobic pocket 21 composed of the hydrophobic tail groups 14. One or more hydrophobic active ingredients 22 may be encapsulated by the micelle 10 in the hydrophobic pocket 21.

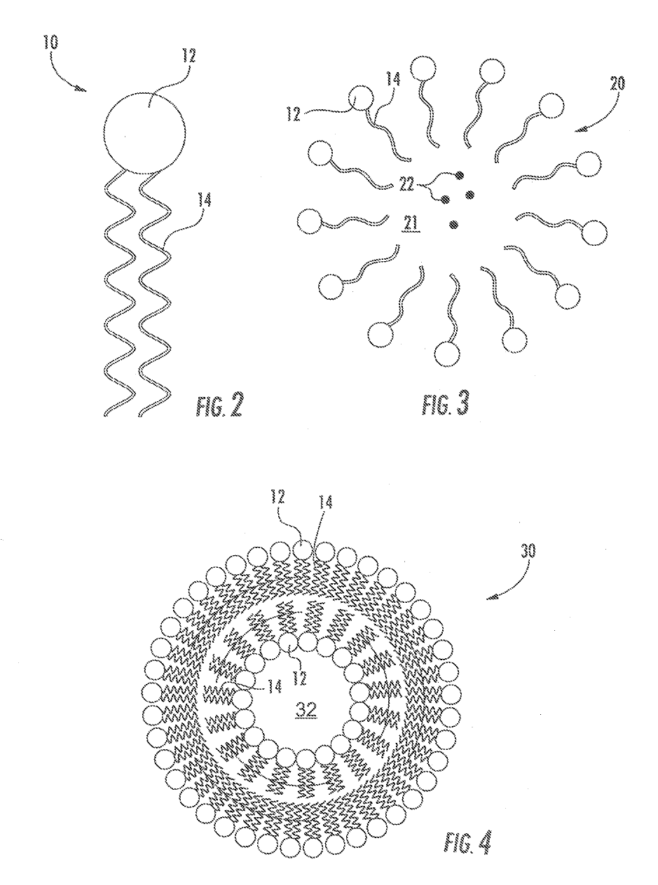

Referring to FIG. 4, a liposome 30 is a vesicle similar to a micelle 20, but composed instead of a plurality of lipid layers. The liposome of FIG. 3 includes a lipid bilayer. Liposomes 30 are capable of encapsulating hydrophobic active ingredients in the hydrophobic tail 14 region of the lipid bilayer and hydrophilic active ingredients in the aqueous pocket 32 at the center of the liposome 30.

Some preferred substances used to form micelles 20 or liposomes 30 include, but are not limited to, both natural and synthetic phosphatidyl-based substances (phospholipids) including lecithins(phosphatidylcholines), hydroxylated lecithin, polyethyleneglycol phospholipid, hydrogenated soy phosphatidylcholine, phosphatidic acid, phosphatidylglycerol, phosphatidylethanolamine, phosphatidylserine, sulfolipids such as sulfoquinovosyl distearoylglycerol, sulfates such as sodium lauryl sulfate, sulfonates such as dioctyl sodium sulfosuccinate, and carboxylates such as sodium deoxycholate, sodium stearate, and sodium oleate. The preferred amount of phospholipid is about 0.0% to about 8.0% w/w and preferably in amounts of about 2% to about 6% w/w, even more preferably about 3.5% to about 4.5% w/w. Hydroxylated lecithin is the preferred phospholipid.

In embodiments in which the oral thin film 8 composition comprises a colloidal suspension, the hydrophobic carriers are lipophilic particles or droplets suspended in an aqueous medium. Such hydrophobic carriers can be formed from almond oil, argan oil, avocado oil, canola oil, cashew oil, castor oil, coconut oil, cod liver oil, colza oil, corn oil, cottonseed oil, fish oil, grapeseed oil, hazelnut oil, hemp oil, linseed oil (flaxseed oil), macadamia oil, marula oil, mongongo nut oil, mustard oil, olive oil, palm oil (palm kernel oil), peanut oil, pecan oil, perilla oil, pine nut oil, pistachio oil, poppy seed oil, pumpkin seed oil, grapeseed oil, rice bran oil, safflower oil, sesame oil, soybean oil, sunflower oil, tea seed oil, walnut oil, watermelon seed oil, and combinations thereof. The preferred amount of hydrophobic carrier forming agent is about 0.0% to about 15% w/w and preferably in amounts of about 2% to about 10% w/w, even more preferably about 3.5% to about 4.5% w/w. Grapeseed oil is particularly preferred due to its relatively low viscosity.

Optionally, the compositions include one or more emulsifiers to prevent the hydrophobic carriers from agglomerating and settling into a continuous oil phase. The use of an emulsifier is more important in the colloidal suspensions. Suitable emulsifiers include, but are not limited to, lecithin, hydroxylated lecithin, sodium stearyl lactylate, cetearyl alcohol, polysorbates, polyoxyethylene ethers, polyethylene glycol, anisolic compounds, and any conventional emulsifier. A preferred concentration range of emulsifier is approximately 0.0% w/w to approximately 20% w/w and preferably in amounts of about 4% to about 14% w/w. Glycerin is the preferred emulsifier.

Optionally, the compositions may include water soluble polymers. Water soluble polymers refer to any polymeric composition that is soluble in aqueous solution, and include, without limitation, cellulose derivatives such as hydroxyethylcellulose, methylcellulose, and hydroxypropyl-methylcellulose, agarose, hyaluronan, acacia, amylase, casein, carboxymethyl cellulose, carboxyvinyl polymer, carrageenans, chitosan, collagen, dextrin, elsinan, gelatin, guar gum, gum Arabic, hydroxypropylated high amylase starch, levan, locust bean gum, methylmethacrylate copolymer, pectin, polyacrylic acid, polyethylene, polyvinyl alcohol, polyvinyl pyrrolidone, pullulan, sodium alginate, soy protein isolate, gum tragacanth, and whey. The preferred amount of water soluble polymer is about 0.0% to about 25% w/w and preferably in amounts of about 4% to about 20% w/w, even more preferably about 9% w/w. Gelatin is a preferred water soluble polymer, and pectin is a more preferred water soluble polymer.

Optionally, the compositions include one or more mucoadhesive polymers. Mucoadhesive polymers refer to any polymer having a desirable in vivo mucosal absorption rate, level of safety, and rate of degradation. Examples of mucoadhesive polymers include, without limitation, alginate, chitosan, collagen, gelatin, hyaluronate, poly(ethyleneimine), poly(2-hydroxyethyl methacrylate), poly(acrylic acid), poly(ethylene oxide), and poly(L-lysine). The preferred amount of mucoadhesive polymer is about 0.0% to about 25% w/w and preferably in amounts of about 8% to about 20% w/w. Gelatin is the preferred mucoadhesive polymer.

Optionally, one or more additional ingredients that are found in conventional pharmaceuticals or nutritional supplements for the preparation of a final dosage form as is readily understood in the art can be added to the composition. These include, but are not limited to, excipients, diluents, disintegrants, solvents, processing aids, buffering agents, colorants, flavorings, binders, carriers, gelling agents, suspending agents, sweetening agent, anti-adherents, preservatives, emulsifiers, antioxidants, plasticizers, surfactants, viscosity agents, enteric agents, wetting agents, thickening agents, stabilizing agents, solubilizing agents, bioadhesives, film forming agents, essential oils, emollients, dissolution enhancers, dispersing agents, or combinations thereof.

Optionally, the compositions include one or more preservatives for preventing the composition from spoiling. Suitable preservatives include, but are not limited to, antimicrobial preservatives and antioxidants. Examples include sorbic acid and its salts, benzoic acid and its salts, calcium propionate, sodium nitrite, sodium nitrate, sulfites, sulfur dioxide, sodium bisulfite, potassium hydrogen sulfite, disodium EDTA, butylated hydroxyanisole, butylated hydroxytoluene, tert-butylhydroquinone, propyl gallate, ethanol, and methylchloroisothiazolinone. Sodium benzoate is a particularly preferred preservative.

Suitable sweeteners contemplated for inclusion in the composition include both natural and artificial sweeteners. For example, water-soluble sweetening agents such as monosaccharides, disaccharides and polysaccharides such as corn syrup solids, dihydrochalcones, fructose, galactose, glucose (dextrose), glycyrrhizin, invert sugar, maltose, maltodextrin, mannose, monellin, partially hydrolyzed starch, ribose, steviosides, sucrose, sucralose, Treha® trehalose, Xtend® sucromalt, and xylose. Water-soluble artificial sweeteners contemplated are those such as the soluble saccharin salts, i.e., sodium or calcium saccharin salts, cyclamate salts, salts of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and any other sweeteners known in the art. Additionally, dipeptide based sweeteners are also optionally included, such as L-aspartyl-L-phenylalanine methyl ester (aspartame), L-alpha-aspartyl-N-(2,2,4,4-tetramethy 1-3-thietany 1)-D-alaninamide hydrate, methyl esters of L-aspartyl-L-phenylglycerin and L-aspartyl-L-2,5, dihydrophenyl-glycine, L-aspartyl-2,5-dihydro-L-phenylalanine, and L-aspartyl-L(1-cyclohexyen)-alanine. The preferred sweetners are acesulfame potassium, sucralose, and maltodextrin.

The invention is made preferably according to the method that is now described. The inventors have overcome the difficulty of providing a film that encapsulates the active ingredients in a colloid, yet has an appropriate moisture content such that the end product is not too dry and brittle yet not so moist as to stick to adjacent film strips when packaged. Purified water is heated to 78-85° C. (172-185° F.) in a mixing kettle and transferred to a mixer. Cocoa butter and soy lecithin are added into the mixer and blended for 1-2 minutes. D-amino acids are added to the solution and blended to the appearance of homogeneity. Peppermint oil, preferably Crystal White®, and glycerin are then added to solution and blended for approximately 1-2 minutes.

Grapeseed oil and glycerin are mixed in an aqueous solution heated to a temperature of approximately 70° C. to approximately 90° C. or, more preferably, approximately 78° C. to approximately 85° C. The one or more pharmaceutically active ingredients, menthol, and pectin are then added to the mixture to form a blend. The blend is finally agitated until it becomes homogeneous. Sweetener, Monoammonium Glycyrrhizinate, Acesulfame Potassium, Microcrystalline Cellulose, coloring, and pectin are added to the solution and blended thoroughly until homogenous.

The present invention has been described hereinabove, and unless otherwise defined, all technical language used herein is intended to have the same meaning as commonly understood in the art to which the invention pertains at the time of its filing. Although various methods and materials similar to those described herein can be used in the practice or testing of the present invention, only some of the suitable methods and materials are described. The skilled should understand that the methods and materials used and described are examples and may not be the only ones suitable for use in the invention.

Accordingly, this invention may be embodied in many different forms and should not be construed as limited to the embodiments set forth herein. Rather, these illustrated embodiments are provided so that this disclosure will be thorough, complete, and will fully convey the scope of the invention to those skilled in the art. Therefore, in the specification set forth above there have been disclosed typical preferred embodiments of the invention, and although specific terms are employed, the terms are used in a descriptive sense only and not for purposes of limitation. The invention has been described in some detail, but it will be apparent that various modifications and changes can be made within the spirit and scope of the invention as described in the foregoing specification and as defined in the appended claims.

Any element in a claim that does not explicitly state “means for” performing a specified function, or “step for” performing a specified function, is not to be interpreted as a “means” or “step” clause as specified in 35 U.S.C. §112, ¶6. In particular, the use of “step of” in the claims herein is not intended to invoke the provisions of 35 U.S.C. §112, ¶6.

The following are presented by way of example:

### EXAMPLE 1

An oral thin film is prepared by first creating a 150 g batch of material with the following weight percent composition:

TABLE 1

| Material | Weight % |
|---|---|
| Water | 70.0-90.0 |
| Pectin | 4.0-9.0 |
| Grapeseed Oil | 0.5-4.0 |
| Glycerin | 0.0-6.0 |
| Hydroxylated Lecithin | 0.0-4.0 |
| Acesulfame Potassium (Ace-K) | 0.5-3.5 |
| Sucralose | 0.5-3.5 |
| Lemon Flavoring (Natural Extract) | 1.0-5.0 |

To this mixture is added 0.6-12.0 mg D-leucine, 0.9-18.0 mg D-tryptophan, 0.7-14.0 mg D-methionine, and 0.8-16.0 mg D-tyrosine. The material is blended until uniform, then spread on three 30×40 cm glass plates and warmed in an 85-110 degree Celsius oven until dry. The film is then powdered with talc, cellulose, and sucralose, and then cut into 21×38 mm strips, about 100 mg weight each.

### EXAMPLE 2

Chewing gum is prepared by warming and mixing a 150 g batch of material with the following weight percent composition:

TABLE 2

| Material | Weight % |
|---|---|
| Chewing Gum Base | 80.0-95.0 |
| Acesulfame Potassium (Ace-K) | 0.5-5.0 |
| Sucralose | 0.5-5.0 |
| Lemon Flavoring (Natural Extract) | 1.0-5.0 |

To this mixture is added 0.6-12.0 mg D-leucine, 0.9-18.0 mg D-tryptophan, 0.7-14.0 mg D-methionine, and 0.8-16.0 mg D-tyrosine. The material is blended until uniform, then spread on two 30×40 cm glass plates and allowed to cool. The gum is then cut into 20×75 mm rectangular strips.

### EXAMPLE 3

Oral gel is prepared by mixing a 200 g batch of material with the following weight percent composition:

TABLE 3

| Material | Weight % |
|---|---|
| Water | 5.0-15.0 |
| Cargill Treha ® (trehalose) | 15.0-25.0 |
| Cargill XTendSucromalt ® | 20.0-50.0 |
| Glycerin | 1.0-10.0 |
| Grapeseed Oil | 1.0-10.0 |
| Hydroxylated Lecithin | 0.0-4.0 |
| Maltodextrin | 5.0-20.0 |
| Acesulfame Potassium (Ace-K) | 0.5-5.0 |
| Sucralose | 0.5-5.0 |
| Lemon Flavoring (Natural Extract) | 1.0-5.0 |

To this mixture is added 0.3-6.0 mg D-leucine, 0.4-8.0 mg D-tryptophan, 0.3-6.0 mg D-methionine, and 0.4-8.0 mg D-tyrosine. The material is blended until uniform. FD&C Yellow #5 or another coloring agent is added until a commercially marketable color is imparted.

### EXAMPLE 4

Gummi-type chewable vehicle is prepared by mixing a 300 g batch of material with the following weight percent composition:

TABLE 4

| Material | Weight % |
|---|---|
| Water | 45.0-70.0 |
| Sucrose | 14.0-30.0 |
| Gelatin | 8.0-20.0 |
| Lemon Flavoring (Natural Extract) | 1.0-5.0 |

The solid materials are added to room temperature water then warmed until all the solid materials dissolve. The mixture is cooled to about 40 degrees C., to which is then added 1.8-36.0 mg D-leucine, 2.8-56.0 mg D-tryptophan, 2.0-40.0 mg D-methionine, and 2.5-50.0 mg D-tyrosine. The material is blended until the amino acids are dissolved. FD&C Yellow #5 or another coloring agent is added until a commercially marketable color is imparted. The mixture is poured into 1×18 inch molds then cooled in a freezer for 15 minutes. The material is removed from the mold and cut into rhombohedrons that weigh about 2.2 g each.

### EXAMPLE 5

Toothpaste is prepared by mixing a 300 g batch of material with the following weight percent composition:

TABLE 5

| Material | Weight % |
|---|---|
| Calcium Phosphate | 65.0-80.0 |
| PEG-12 | 0.0-16.0 |
| Glycerin | 5.0-14.0 |
| Carboxymethylcellulose sodium | 0.0-8.0 |
| Carrageenan | 0.0-10.0 |
| Lemon Flavoring | 1.0-5.0 |
| Sucralose | 0.5-5.0 |
| Acesulfame Potassium | 0.5-5.0 |

The solid materials are blended together. In a separate vessel, the liquid materials are blended together. To the liquid materials are added 1.8-36.0 mg D-leucine, 2.8-56.0 mg D-tryptophan, 2.0-40.0 mg D-methionine, and 2.5-50.0 mg D-tyrosine. These are blended until dissolved or evenly dispersed. The liquid materials are poured into the solid materials, stirring until the final mixture has a paste consistency.

### EXAMPLE 6

Mouthwash/mouth rinse/oral spray is prepared by mixing a 300 g batch of material with the following weight percent composition:

TABLE 6

| Material | Weight % |
|---|---|
| Water | 75.0-90.0 |
| Glycerin | 5.0-18.0 |
| Lemon Flavoring | 1.0-5.0 |
| Sucralose | 0.5-5.0 |
| Acesulfame Potassium | 0.5-5.0 |

Liquid materials are blended together, and then the solid materials are added, stirring until completely dissolved. To the mixture are added 1.8-36.0 mg D-leucine, 2.8-56.0 mg D-tryptophan, 2.0-40.0 mg D-methionine, and 2.5-50.0 mg D-tyrosine. The mixture is stirred until dissolution is complete.

The products incorporating D-amino acids or D,L-amino acids may be used, as indicated for human use, also in animals to reduce or eliminate dental plaque. Alternatively, the products may be modified to maximize efficacy for the reduction or elimination of dental plaque in animals and to maximize palatability for animals.

Many modifications and other embodiments of the invention will come to the mind of one skilled in the art having the benefit of the teachings presented in the foregoing descriptions and tables. Therefore, it is to be understood that the invention is not to be limited to the specific embodiments disclosed, and that modifications and alternate embodiments are intended to be included within the scope of the claims supported by this specification.

What is claimed is:

1. A thin film composition for oral administration that adheres to and dissolves in a mouth of a user, wherein the thin film is a monolayer, water-soluble solid comprising at least one D-amino acid.

2. The thin film composition according to claim 1, wherein the at least one D-amino acid comprises D-leucine, D-tryptophan, D-methionine, and D-tyrosine.

3. The thin film composition according to claim 2, comprising:
about 0.01% to about 1.0% w/w D-leucine;
about 0.01% to about 1.0% w/w D-tryptophan;
about 0.01% to about 1.0% w/w D-methionine; and
about 0.01% to about 1.0% w/w D-tyrosine.

4. The thin film composition according to claim 1, wherein the at least one D-amino acid is contained in a plurality of hydrophobic carriers dispersed throughout the thin film.

5. The thin film composition according to claim 4, wherein the hydrophobic carriers comprise oil and the composition further comprises a phospholipid, an emulsifier, and a water soluble polymer.

6. The thin film composition according to claim 5, wherein the oil is grapeseed oil.

7. The thin film composition according to claim 5, wherein the oil is approximately 0.5% to approximately 4% w/w of the composition, the phospholipid is approximately 0% to approximately 4% w/w of the composition, the emulsifier is approximately 0% to approximately 6% w/w of the composition, and the water soluble polymer is approximately 4% to approximately 9% w/w of the composition.

8. The thin film composition according to claim 7, wherein the phospholipid is hydroxylated lecithin.

9. The thin film composition according to claim 7, wherein the emulsifier is glycerine.

10. The thin film composition according to claim 7, wherein the water soluble polymer is pectin.

11. The thin film composition according to claim 7, wherein the at least one D-amino acid is approximately 0.1% to approximately 4% w/w of the composition.

12. The thin film composition according to claim 7, further comprising at least one sweetener being approximately 1% to about 7% w/w of the composition and a flavoring being approximately 1% to about 5% w/w of the composition.

13. The thin film composition according to claim 12, wherein the sweetener is chosen from the group comprising acesulfame potassium, sucrose, and sucralose.

14. A thin film composition for oral administration that adheres to and dissolves in a mouth of a user, wherein the thin film is a monolayer, water-soluble solid comprising:
at least one D-amino acid being approximately 0.1% to approximately 4% w/w of the composition, the at least one D-amino acid being contained in a plurality of hydrophobic carriers dispersed throughout the thin film;
grapeseed oil being approximately 0.5% to approximately 4% w/w of the composition;
hydroxylated lecithin being approximately 0% to approximately 4% w/w of the composition;
glycerin being approximately 0% to approximately 6% w/w of the composition;
pectin being approximately 4% to approximately 9% w/w of the composition;
water being approximately 65% to approximately 95% w/w of the composition; and
sweetener being approximately 0.5% to approximately 7% w/w of the composition.

15. The thin film composition according to claim 14, wherein the at least one D-amino acid comprises D-leucine, D-tryptophan, D-methionine, and D-tyrosine.

16. The thin film composition according to claim 14, comprising:
about 0.025% to about 1.0% w/w D-leucine;
about 0.025% to about 1.0% w/w D-tryptophan;
about 0.025% to about 1.0% w/w D-methionine; and
about 0.025% to about 1.0% w/w D-tyrosine.

17. A method of reducing dental plaque in a subject, the method comprising placing the thin film composition of claim 1 into a mouth of the subject.
